(19) United States
(12) Patent Application Publication
(10) Pub. No.: US 2018/0064351 A1
(43) Pub. Date: Mar. 8, 2018

(54) BIOLOGICAL INFORMATION DETECTION DEVICE, DETECTION DEVICE, AND ELECTRONIC APPARATUS

(71) Applicant: SEIKO EPSON CORPORATION, Tokyo (JP)

(72) Inventor: Kazuyoshi KEGASAWA, Hara-mura (JP)

(73) Assignee: SEIKO EPSON CORPORATION, Tokyo (JP)

(21) Appl. No.: 15/689,619

(22) Filed: Aug. 29, 2017

(30) Foreign Application Priority Data
Sep. 5, 2016 (JP) 2016-172670

(51) Int. Cl. A61B 5/026; H05B 37/02; A61B 5/00; A61B 5/024; A61B 5/1455

(52) U.S. Cl. CPC A61B 5/0261; H05B 37/0227; A61B 5/14551; A61B 5/02416; A61B 5/681

(57) ABSTRACT

A biological information detection device includes: a light-emitting unit that emits light to a subject; a light-receiving unit that receives reflected light or transmitted light from the subject; and a current control unit that supplies the light-emitting unit with a current signal for causing the light-emitting unit to emit the light. For a first period of a light emission period of the light-emitting unit, the current control unit supplies the light-emitting unit with the current signal with a current value greater than for a second period of the light-emission period.

### CROSS-REFERENCES TO RELATED APPLICATIONS

This application claims priority to Japanese Patent Application No. 2016-172670, filed Sep. 5, 2016, the entirety of which is herein incorporated by reference.

### BACKGROUND

### 1. Technical Field

The present invention relates to a biological information detection device, a detection device, and an electronic apparatus.

### 2. Related Art

There are known electronic apparatuses (optical meters) in which light sources such as light-emitting diodes (LEDs) emit light to radiate the light to subjects and light-receiving units such as photodiodes (PDs) receive reflected light or transmitted light. In optical meters, various physical amounts are measured based on light reception results in the light-receiving units.

In optical meters, it is necessary to increase luminance of light sources in order to ensure signal levels sufficient for measurement. However, the increase in the luminance of the light sources leads to an increase in power consumption. In order to suppress the increase in the power consumption, schemes of causing the light sources to intermittently emit light by pulse driving and performing sampling only at the time of emitting the light are frequently used.

For example, JP-A-2016-67406 discloses a measurement apparatus applying a pulse current to a light-emitting unit and a scheme of switching two detection modes in which current values of the pulse currents are different.

To suppress a fluctuation in a sampling value by noise, the sampling value is passed through an LPF (or a BPF) before the sampling to limit a frequency bandwidth in many cases. Therefore, since a pulse-modulated signal becomes blunt by filter processing, it takes some times to reach a desired value. Thus, there is a problem that a pulse width causing a light source to emit light is lengthened.

### SUMMARY

An advantage of some aspects of the invention is to provide a biological information detection device, a detection device, and an electronic apparatus appropriately controlling a current value to be supplied to a light-emitting unit within a light emission period.

Another advantage of some aspects of the invention is to provide a biological information detection device, a detection device, and an electronic apparatus reducing power consumption by controlling a current value to be supplied to a light-emitting unit within a light emission period.

An aspect of the invention relates to a biological information detection device including: a light-emitting unit that emits light to a subject; a light-receiving unit that receives reflected light or transmitted light from the subject; and a current control unit that supplies the light-emitting unit with a current signal for causing the light-emitting unit to emit the light. For a first period of a light emission period of the light-emitting unit, the current control unit supplies the light-emitting unit with the current signal with a current value greater than for a second period which is a period after the first period of the light emission period.

According to the aspect of the invention, current control is performed such that the current value is relatively larger for the first period of the light emission period than for the second period after the first period. In this way, the signal which is a signal based on a light reception result of the light-receiving unit and is a sampling target reaches a desired value in a relatively short time. Therefore, it is possible to shorten the light emission period necessary to perform stable sampling. Thus, for example, it is possible to reduce power consumption.

In the aspect of the invention, the biological information detection device may further include a detection unit that performs a process of detecting a signal from the light-receiving unit. The detection unit may include a filter unit. The current control unit may supply the current signal including a frequency component higher than a cutoff frequency of the filter unit to the light-emitting unit for the first period.

With this configuration, the high-frequency component reduced by the filter unit can be included in the current signal, and thus it is possible to compensate bluntness of the waveform by the filter unit.

In the aspect of the invention, the filter unit may be a lowpass filter or a bandpass filter. The cutoff frequency may be a cutoff frequency of the lowpass filter or a high-frequency-side cutoff frequency of the bandpass filter.

With this configuration, when a lowpass filter or a bandpass filter is used as the filter unit, appropriate current control is possible.

In the aspect of the invention, when τ is a time constant of the filter unit, a length of the light emission period may be equal to or less than P (where P is a positive equal to or less than 4)×τ.

With this configuration, it is possible to further shorten the light emission period than in a scheme of the related art and it is possible to reduce power consumption.

In the aspect of the invention, when Ia is the current value for the second period, a total current value for the light emission period may be less than a total current value when the current signal of which the current value is Ia flows for a period with a length of 5×τ.

With this configuration, it is possible to further reduce power consumption than in a scheme of supplying a current signal with a current value constant for the light emission period.

In the aspect of the invention, when TL is a length of the light emission period, a length of the first period may be equal to or less than TL/Q (where Q is 2 or more).

With this configuration, since the first period in which the current value of the current signal is relatively larger can be shortened, it is possible to improve an effect of reducing power consumption.

In the aspect of the invention, the current signal may be a current signal of which the current value is constant for the first period and is greater than the current value for the second period.

With this configuration, through the current control in which the current value is changed in two stages for the first and second periods, it is possible to reduce power consumption.

In the aspect of the invention, the current signal may have a peak value at a predetermined timing in the first period and may be a current signal of which the peak value is greater than the current value for the second period.

With this configuration, through the current control in which the peak value is provided for the first period and the current value decreases from the peak value for the second period, it is possible to reduce power consumption.

In the aspect of the invention, the current signal may be a current signal of which a current variation value per unit time in falling from the peak value is less than a current variation value per unit time in rising to the peak value.

With this configuration, a change in the current value in the falling can be set to be gentle. Therefore, for example, even when there is an influence of parasitic capacitance or parasitic inductance, it is possible to prevent the signal from vibrating and perform stable sampling.

In the aspect of the invention, the current control unit may include a D/A conversion circuit that performs D/A conversion on a current setting value for setting a waveform of the current signal for the first and second periods, and a current supply circuit that outputs a current corresponding to an output voltage of the D/A conversion circuit as the current signal.

With this configuration, since the current signal can be supplied by the D/A conversion circuit and the current supply circuit, the current value of the current signal can be controlled based on the current setting value which is digital data.

Another aspect of the invention relates to a detection device including: a light-emitting unit that emits light to a target object; a light-receiving unit that receives reflected light or transmitted light from the target object; and a current control unit that supplies the light-emitting unit with a current signal for causing the light-emitting unit to emit the light. The current control unit includes a D/A conversion circuit that performs D/A conversion on a current setting value for setting a waveform for a light emission period of the light-emitting unit for a D/A conversion period equal to or less than ½ of the light emission period, and a current supply circuit that outputs a current corresponding to an output voltage of the D/A conversion circuit as the current signal.

According to the aspect of the invention, in the detection device in which the current signals to the light-emitting unit are supplied by the D/A conversion circuit and the current supply circuit, the D/A conversion period of the D/A conversion circuit is a period with a length equal to or less than ½ of the light emission period. In this way, it is possible to switch the current value of the current signal at a plurality of stages. Thus, for example, it is possible to flexibly control the waveform of the current signal.

Still another aspect of the invention relates to an electronic apparatus including the biological information detection device or the detection device.

### BRIEF DESCRIPTION OF THE DRAWINGS

The invention will be described with reference to the accompanying drawings, wherein like numbers reference like elements.

FIG. 1 is a diagram illustrating waveform examples of a current signal and an output signal according to a scheme of the related art.

FIG. 2 is a diagram illustrating a configuration example of a biological information detection device.

FIG. 3 is a diagram illustrating configuration example of a light-emitting unit and a current control unit.

FIG. 4 is a diagram illustrating a configuration example of a light-receiving unit and a detection unit.

FIG. 5 is a diagram illustrating a configuration example of a transimpedance amplifier.

FIG. 6 is a diagram illustrating waveform examples of a current signal and an output signal according to a first embodiment.

FIG. 7 is a diagram illustrating a relation between N and a reaching ratio.

FIG. 8 is a diagram illustrating waveform examples of a current signal and an output signal according to the first embodiment when there is an influence of a parasitic capacitance or parasitic inductance.

FIG. 9 is a diagram illustrating expanded waveform examples of a current signal and an output signal according to the first embodiment when there is an influence of a parasitic capacitance or parasitic inductance.

FIG. 10 is a diagram illustrating waveform examples of a current signal and an output signal according to a second embodiment.

FIG. 11 is a diagram illustrating waveform examples of a current signal and an output signal according to the second embodiment when there is an influence of a parasitic capacitance or parasitic inductance.

FIG. 12 is a diagram illustrating expanded waveform examples of a current signal and an output signal according to the second embodiment when there is an influence of a parasitic capacitance or parasitic inductance.

FIG. 13 is a diagram illustrating an outer appearance example of a wearable apparatus which is an electronic apparatus including a biological information detection device.

FIG. 14 is a diagram illustrating an outer appearance example of a wearable apparatus which is an electronic apparatus including a biological information detection device.

FIG. 15 is a diagram illustrating a configuration example of a detection device.

FIG. 16 is a perspective view illustrating main units of a printing apparatus which is an electronic apparatus including a detection device.

### DESCRIPTION OF EXEMPLARY EMBODIMENTS

Hereinafter, embodiments will be described. The embodiments to be described below do not inappropriately limit content of the invention described in the appended claims. All of the configurations to be described in the embodiments may not be indispensable constituent elements of the invention.

### 1. Scheme of Embodiment

First, a scheme of an embodiment will be described. In the related, there are known light sensor modules including light-emitting units and a light-receiving units or various devices including the light sensor modules. For example, a light sensor module is used for a biological information detection device that acquires biological information when a light-emitting unit radiates light to a subject (organism) from a light-emitting unit and a light-receiving unit receives reflected light or transmitted light from the organism. Here, the biological information is information indicating an organism activity state of the subject. Hereinafter, an example in which reflected light is used will be described. However, transmitted light can also be considered to be used instead.

In the biological information detection device, light with a wavelength bandwidth easily absorbed by blood (in a narrow sense, hemoglobin included in blood) is radiated from the light-emitting unit. When a blood flow rate is high and an amount of hemoglobin is also large, an absorption amount of light is large and the intensity of reflected light becomes small. In contrast, when a blood flow rate is low and an amount of hemoglobin is also small, an absorption amount of light is small and the intensity of reflected light becomes large. In this case, since a variation in a signal (AC component) from the light-receiving unit indicates a variation in the blood flow rate, pulse wave information can be obtained based on a signal from the light-receiving unit in the biological information detection device.

Alternatively, the light-emitting unit may be configured to radiate light with a first wavelength bandwidth having a relatively great absorption coefficient of oxidized hemoglobin and light with a second wavelength bandwidth having a relatively great absorption coefficient of reduced hemoglobin. In this case, a ratio of oxidized hemoglobin to reduced hemoglobin in blood can be estimated using a light reception signal of reflected light caused by the light with the first wavelength bandwidth and a light reception signal of reflected light caused by the light with the second wavelength bandwidth. That is, the biological information detection device can obtain oxygen saturation (in a narrow sense, arterial oxygen saturation SpO2) in blood as biological information based on a signal from the light-receiving unit.

In the biological information detection device, a scheme of intermittently operating the light-emitting unit is broadly used to reduce power consumption of the light-emitting unit (an LED or a semiconductor laser). For example, in JP-A-2016-67406, the light-emitting unit is used to emit light in a pulse form.

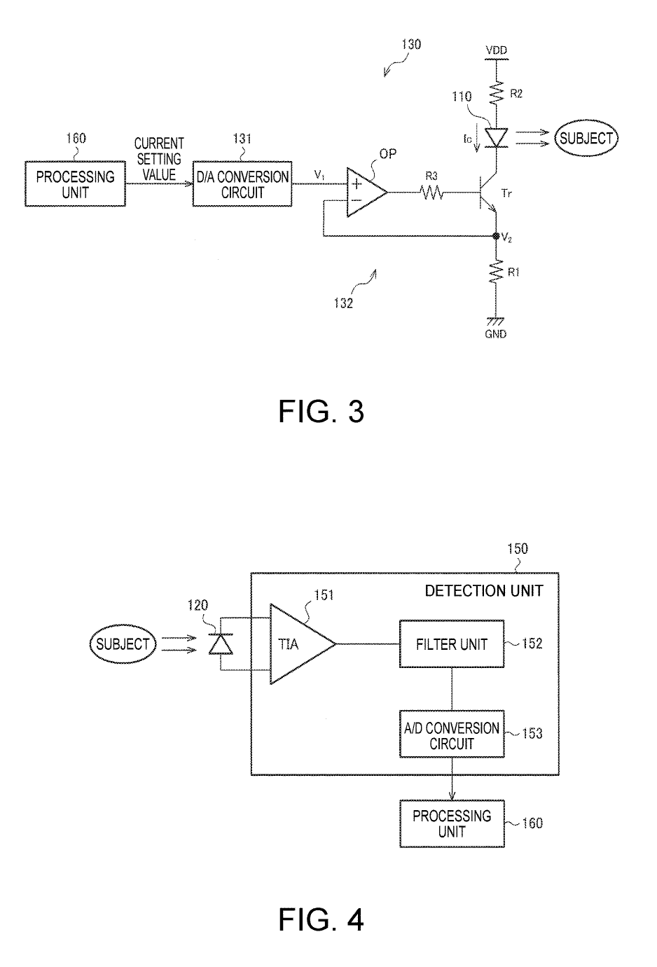

When a current signal with a current value Ia is supplied to the light-emitting unit, radiation light with an intensity La according to Ia is radiated from the light-emitting unit and light with an intensity Lb according to the intensity La is reflected as reflected light from a target object (subject). The light-receiving unit receives the reflected light with the intensity Lb and outputs a current signal with the current value Ib according to the intensity Lb. As will be described below with reference to FIG. 3, when the biological information detection device includes a transimpedance amplifier that performs voltage conversion and amplification on a current signal from the light-receiving unit, the transimpedance amplifier outputs an analog voltage signal serving as the voltage value Vb according to the current value Ib. The analog voltage signal is converted (sampled) into a digital signal by an A/D conversion circuit to become a target to be processed by a processing unit.

As understood from the foregoing flow, the voltage value Vb of the signal (hereinafter referred to as an output signal) sampled by the biological information detection device is ideally decided according to the current value Ia of a current signal supplied to the light-emitting unit. In other words, when a current signal with the current value Ia is supplied, the intensity of the output signal is expected to be a predetermined desired intensity (the voltage value Vb). It is not desirable that an actual signal intensity considerably deviates from the desired intensity. For example, this is because although a large current flows in the light-emitting unit, there is a concern of the signal intensity of the output signal not reaching a sufficient level.

The intensity Lb of the reflected light is information changed due to a target object state as well as the intensity La of the radiation light. For example, in the example of the above-described pulse wave information, when the blood flow rate in a blood vessel is changed by pulsation, the value of Lb is changed despite of the constant La. Even in this case, when the target state is constant, it is important for a voltage value by the current value Ia to be nearly a constant value. This is because when a large change in the voltage value of the output signal with respect to the current signal of the current value Ia is allowed despite the constant target state, it is not distinguished that the change in the voltage value is caused due to the side of the subject or due to the side of the biological information detection device, and thus detection precision of the biological information may deteriorate.

At this time, a problem is a filter unit (filter circuit) installed on the front stage of the A/D conversion circuit. The filter unit herein can be considered to be an anti-aliasing filter that suppresses an aliasing error of a high-frequency component or can also be considered to be a noise reduction filter that suppresses a fluctuation in a sampling value by high-frequency noise. In either case, since the filter unit is a filter that reduces a high-frequency component, an output signal waveform which is originally the same waveform as the current signal supplied to the light-emitting unit may become blunt.

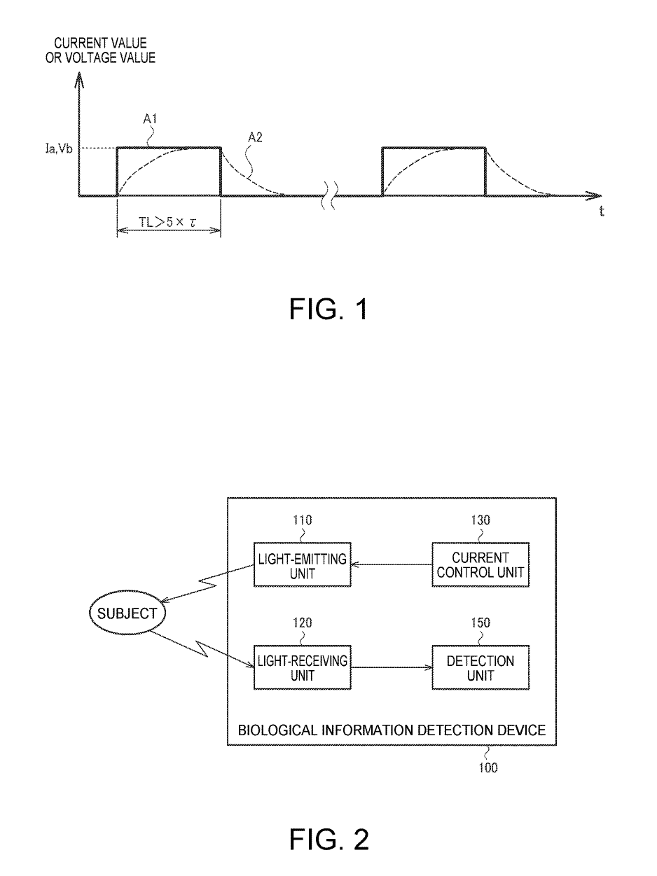

FIG. 1 is a diagram illustrating waveform of a current signal (driving current) and an output signal (output voltage) according to a scheme of the related art. A1 indicates a current signal waveform and A2 indicates an output signal waveform. In FIG. 1, the horizontal axis represents a time, the vertical axis in regard to A1 indicates a current value, and the vertical axis in regard to A2 indicates a voltage value. As illustrated in FIG. 1, since the output signal becomes blunt through filter processing, it takes some time to reach Vb which is a desired value. For example, when τ is a time constant of a filter, a temporal change of an output signal corresponding to A2 is expressed by Expression (1) below. In a simple lowpass filter configured to includes a capacitor C connected in parallel to an input signal and a resistor R connected in series to the input signal, τ=RC is satisfied. In addition, those skilled in the art can easily understand that the time constant τ can be decided depending on the configuration of the filter.

$$V ( t ) = Vb \times ( 1 - exp ( - \frac{t}{τ} ) ) \qquad ( 1 )$$

Therefore, in the example of FIG. 1, a time longer than 5×τ (for example, 6×τ) elapses, a signal is stabilized, and sampling is performed. This is because in the example of Expression (1) above, the voltage value of the output signal is greater than 0.99×Vb after the elapse of 5×τ, and thus is sufficiently close to Vb which is a desired value. In this case, as indicated by A1, the light-emitting unit has to continuously emit light for a time longer than 5×τ, and thus an effect of reducing power consumption by intermittent driving may be lowered.

However, as illustrated in FIG. 2, a biological information detection device 100 according to the embodiment includes a light-emitting unit 110 that emits light to a subject, a light-receiving unit 120 that receives reflected light or transmitted light from the subject, and a current control unit 130 that supplies the light-emitting unit 110 with a current signal for causing the light-emitting unit 110 to emit the light. The current control unit 130 supplies the light-emitting unit 110 with a current signal with a larger current value for a first period of a light-emission period of the light-emitting unit 110 than for a second period which is a period after the first period of the light emission period. The biological information detection device 100 may include a detection unit 150. The details of the detection unit 150 will be described below.

The light emission period of the light-emitting unit 110 indicates a period in which the light-emitting unit 110 emits light in sampling performed once on an output signal. The light emission period may be a period from light emission start (current signal supply start) to light emission end (current signal supply end) of light emission performed once. The light emission period includes the first and second periods and the first period is a period temporally previous to the second period. Specifically, the end point of the first period is the same timing as the start point of the second period or a timing temporally previous to the second period. A length T1 of the first period may not be equal to a length T2 of the second period. A third period different from the first and second periods may be provided within the light emission period.

As illustrated in FIG. 1, the output signal (A2) after the filter processing is a signal that has a voltage value approaching Vb over time setting the voltage value Vb corresponding to the current value Ia as a target. Accordingly, when a current signal with a sufficiently large current value for the first period is supplied, a target value for the first period is a voltage value sufficiently greater than Vb. In an example to be described below with reference to FIG. 6, the current value for the first period is N×Ia (where N is a value greater than 1) and a target value of the output signal for the first period is N×Vb. In order for the voltage value to approach N×Vb, a time of about 5×τ is necessary as in the above-described example. However, here, a voltage value approaching Vb suffices, and thus a reaching time can be set to be shorter than 5×τ. A current higher than Ia flows for the first period, but the first period may be short (for example, τ/2 or less). Therefore, current consumption can be further reduced than in the scheme of the related art in FIG. 1.

The target value for the first period is greater than Vb and the voltage value does not converge on Vb. Therefore, even when the first period is provided, it is difficult to control a sampling timing. When the sampling timing is too early, the voltage value does not sufficiently approach Vb. When the sampling timing is too late, the voltage value may be considerably greater than Vb. From this viewpoint, in the embodiment, a second period in which a current value is less than for the first period is provided. Accordingly, since the output signal can be stabilized for the second period, sampling can be performed at high precision. From the viewpoint of stabilization of the output signal, the current signal for the second period is preferably set as a signal of which a current value is fixed to Ia or a signal of which a variation is sufficiently small with respect to Ia.

Hereinafter, a system configuration example of the biological information detection device 100 according to the embodiment will be described in detail. Thereafter, a control example in the current control unit 130 will be described. Finally, specific examples of an electronic apparatus 200 and other electronic apparatuses (300 and 400) according to the embodiment will be described.

### 2. System Configuration Example

FIG. 3 is a diagram illustrating a configuration example of a light-emitting unit 110 and a current control unit 130. As illustrated in FIG. 3, the current control unit 130 includes a D/A conversion circuit 131 that performs D/A conversion on a current setting value for setting the waveform of a current signal for the first and second periods and a current supply circuit 132 that outputs a current corresponding to an output voltage of the D/A conversion circuit 131 as a current signal.

A processing unit 160 outputs a current setting value which is digital data to the D/A conversion circuit 131. The processing unit 160 performs a process on the digital data and can be realized by any of various processors such as a micro-control unit (MCU) or a digital signal processor (DSP). The D/A conversion circuit 131 performs D/A conversion on the current setting value and outputs an analog signal (analog voltage).

The current supply circuit 132 includes an operational amplifier OP, a bipolar transistor Tr (power transistor), and resistors R1 to R3. An output terminal of the D/A conversion circuit 131 is connected to a non-inversion input terminal of the operational amplifier OP. An output terminal of the operational amplifier OP is connected to a base of the bipolar transistor Tr via the resistor R3. An emitter of the bipolar transistor Tr is connected to an inversion input terminal of the operational amplifier OP. The resistor R1 is formed between the emitter of the bipolar transistor Tr and a low-potential-side reference potential GND. The resistor R2 and an LED which is the light-emitting unit 110 are connected in series between a collector of the bipolar transistor Tr and a high-potential-side reference potential VDD. In the example of FIG. 3, one end of the resistor R2 is connected to a terminal to which the high-potential-side reference potential VDD is supplied and an anode of the light-emitting unit 110 (the LED) is connected to the other end of the resistor R2. A cathode of the light-emitting unit 110 (the LED) is connected to the collector of the bipolar transistor Tr.

As illustrated in FIG. 3, inputs to the non-inversion input terminal and the inversion input terminal of the operational amplifier OP become an output voltage of the D/A conversion circuit 131 and a voltage of the emitter, respectively. Accordingly, when V1 is the output voltage of the D/A conversion circuit 131 and V2 is the voltage of the emitter, the two voltages are identical, as equal to each other in Expression (2) below. When the low-potential-side reference potential GND is a ground, Expression (3) below is established. Further, Expression (4) below is derived from Expressions (2) and (3) below.

V1=V2 (2)

V2=R1×Ic (3)

Ic=V1/R1 (4)

A collector current Ic corresponds to a current value of the current signal supplied to the light-emitting unit 110. As understood from Expression (4) above, the current supply circuit 132 can supply a current signal with a current value according to the output voltage V1 of the D/A conversion circuit 131 to the light-emitting unit 110. That is, by controlling the current setting value, it is possible to control V1 output from the D/A conversion circuit 131 and a temporal change waveform of the current value of the current signal decided according to V1.

FIG. 4 is a diagram illustrating a configuration example of the light-receiving unit 120 and the detection unit 150. The light-receiving unit 120 (PD) is connected to a transimpedance amplifier 151. The transimpedance amplifier 151 is connected to a filter unit 152. The filter unit 152 is connected to an A/D conversion circuit 153. The A/D conversion circuit 153 is connected to the processing unit 160.

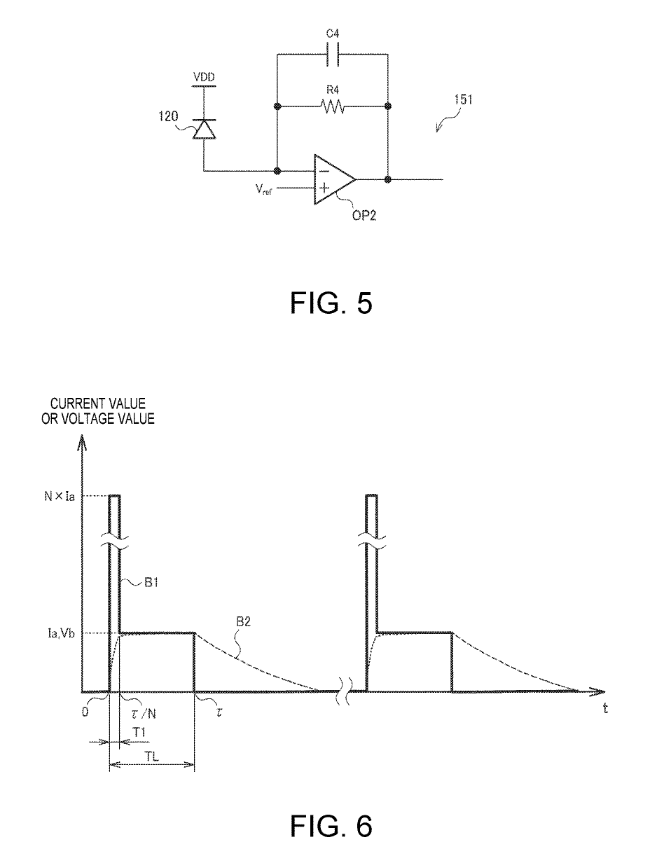

FIG. 5 is a diagram illustrating a detailed configuration example of the transimpedance amplifier 151. The transimpedance amplifier 151 includes an operational amplifier OP2, a resistor R4, and a capacitor C4. An anode of the light-receiving unit 120 (PD) is connected to an inversion input terminal of the operational amplifier OP2. The VDD is supplied to a cathode of the light-receiving unit 120. A signal from the light-receiving unit 120 is input to an inversion input terminal of the operational amplifier OP2. A predetermined reference voltage Vref is input to the non-inversion input terminal of the operational amplifier OP. The reference voltage Vref may be generated, for example, by performing resistive dividing on a voltage between the VDD and the GND.

The VDD and the GND are supplied to two power terminals (not illustrated) of the operational amplifier OP2, and thus the operational amplifier OP operates using signals from the power terminals as power. The resistor R4 and the capacitor C4 are provided in parallel between the output terminal and the inversion input terminal of the operational amplifier OP2. In the foregoing configuration, the operational amplifier OP2 outputs signal obtained by performing voltage conversion and amplification on the output current of the light-receiving unit 120.

The filter unit 152 performs filter processing on an output signal of the transimpedance amplifier 151. The filter unit 152 is a lowpass filter (hereinafter referred to as an LPF) or a bandpass filter (hereinafter referred to as a BPF).

The A/D conversion circuit 153 performs A/D conversion on an analog signal which is an output of the filter unit 152 and outputs digital data which is an A/D conversion result to the processing unit 160. The A/D conversion circuit 153 may be a sequential comparison type A/D conversion circuit, a ΔΣ type A/D conversion circuit, or another A/D conversion circuit, and various modifications thereof are possible.

In FIGS. 3 and 4, the processing unit 160 outputting a current setting value and the processing unit 160 performing a process on an A/D conversion result are assumed to be common, and thus the same reference numeral is given. However, the side of the light-emitting unit 110 (an output side) and the side of the light-receiving unit 120 (a detection side) may be configured to include a processing unit, respectively.

A microcontroller is known to include a D/A conversion circuit, an A/D conversion circuit, and an operational amplifier therein. For example, the processing unit 160, the D/A conversion circuit 131, the operational amplifier OP of the current supply circuit 132, and the A/D conversion circuit 153 in FIGS. 3 and 4 may be configured to be included in a microcontroller. The specific configuration of the biological information detection device 100 may be modified in various forms. For example, a part (for example, the A/D conversion circuit 153) of the foregoing configuration may be provided outside of the microcontroller.

### 3. Control Example in Current Control Unit

Next, a control example of a current signal in the current control unit 130 will be described. As illustrated in FIGS. 2 and 4, the biological information detection device 100 further includes the detection unit 150 that performs a process of detecting a signal from the light-receiving unit 120. The detection unit 150 includes the filter unit 152. As described above with reference to FIG. 1, an output signal waveform becomes blunt by the filter unit 152, and thus a time from supply start of the current signal to the light-emitting unit 110 to sufficient approach of the output signal to the expected voltage value Vb is lengthened. That is, a length TL of the light emission period necessary to perform stable sampling is lengthened, and thus power consumption may increase. Here, the fact that the waveform becomes blunt is equivalent to a reduction in a high-frequency component through filter processing of the filter unit 152. Accordingly, the current control unit 130 according to the embodiment supplies a current signal including a frequency component greater than a cutoff frequency of the filter unit 152 to the light-emitting unit 110 for the first period.

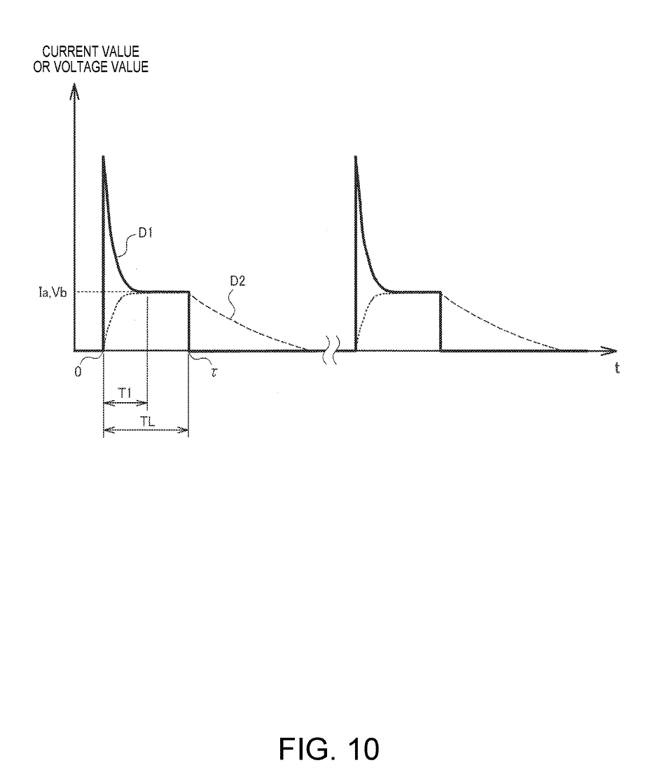

The current signal including the frequency component greater than the cutoff frequency refers to a signal that has large power at a frequency higher than the cutoff frequency when a temporal change waveform of a current for a predetermined period (in a narrow sense, a light emission period) is subjected to frequency conversion. For example, power at a predetermined frequency (>the cutoff frequency) when a current signal according to the embodiment is subjected to frequency conversion is greater than power at the predetermined frequency when a current signal of A1 of FIG. 1 is subjected to frequency conversion. In this way, a high-frequency component compensating bluntness of a waveform can be added to a current signal. As a result, the output signal according to the embodiment has a waveform in which the bluntness is further cancelled than in A2 of FIG. 1 (closer to A1 in shape), and thus a time taken to sufficiently approach the voltage value Vb can be shorter than in A2. A current signal including a high frequency component can be realized by having steep rising and falling of a current value for the light emission period. In the example of FIG. 6, by adding a considerably large rectangular wave of current value for the first period, it is possible to have a frequency component in which rising and falling of the rectangular wave are high. In the example of FIG. 10, by allowing a peak value for the first period, it is possible to have a frequency component in which rising to the peak value and falling from the peak value are high.

The filter unit 152 may be a lowpass filter or a bandpass filter. As described above, this is because the filter unit 152 is an anti-aliasing filter or a high-frequency noise reduction filter and can be realized by a filter that reduces a high-frequency component. In this case, the cutoff frequency of the filter unit 152 is a cutoff frequency of the lowpass filter or a high-frequency-side cutoff frequency of the bandpass filter.

Specifically, when a current value of an input signal increases for the first period, a current change per unit time for a start period and an end period of the first period increases. The start period of the first period is a start period of the light emission period in a narrow sense and the end period of the first period is a boundary period with the second period in a narrow sense. Such control enables the high-frequency component to be included in the input signal. Hereinafter, a first embodiment (FIG. 6) and a second embodiment (FIG. 10) will be described as specific waveform examples.

### 3.1 First Embodiment

In this embodiment, the current control unit 130 performs current control such that a current with a value considerably greater than the target current value Ia flows for the first period and the current value decreases to the target current value Ia for the second period. In this way, even when the light emission period is shortened, an output signal after filter processing quickly increases to be stabilized near a desired value (Vb).

FIG. 6 is a diagram illustrating an example of waveforms of a current signal and an output signal (output voltage) according to the embodiment. B1 indicates a current signal waveform and B2 indicates an output signal waveform. In FIG. 6, the horizontal axis represents a time, the vertical axis in regard to B1 indicates a current value, and the vertical axis in regard to B2 indicates a voltage value. As indicated by B1 of FIG. 6, when N is a number greater than 1 (preferably a number equal to or greater than 2 and, for example, N=20 is set) and T is a time constant of the filter unit 152, a current value for the first period is N×Ia. The first period is a period from a start timing of the light emission period (here, t=0 is set for convenience) to a timing of t=τ/N. The second period is a period from t=τ/N to an end timing (t=τ) of the light emission period.

In this case, B2 which is an output signal after the filter processing rises steeply for the first period (0≦t≦τ/N). Then, the output signal gradually approaches the desired value Vb for the second period (τ/N≦t≦τ). Therefore, when sampling is performed immediately before t=τ, a substantially stable signal can be sampled near the desired value. When a voltage value of the output signal at the timing t is defined as a reaching ratio using the target voltage value Vb as a reference, the reaching ratio is expressed by Expressions (5) and (6), for example. When a voltage value at a predetermined timing is 0.99×Vb, a reaching ratio at that timing is 0.99.

$$\begin{matrix}Reaching ratio ( t ) = N \times ( 1 - exp ( - \frac{t}{τ} ) ) 0 \leq t \leq \frac{τ}{N} & ( 5 ) \\ Reaching ratio ( t ) = N \times ( 1 - exp ( - \frac{t}{τ} ) ) - ( N - 1 ) \times ( 1 - exp ( - \frac{t - \frac{τ}{N}}{τ} ) ) t > \frac{τ}{N} & ( 6 )\end{matrix}$$

For example, when N=20 is set, a reaching ratio at t=τ/20 is about 0.975. Thus, a steep rising can be realized in a very short time. Thereafter, at the time of transition in accordance with Expression (6) above, the reaching ratio at t=τ is 0.99 or more. Therefore, the sampling can be performed with a value sufficiently close to the desired voltage value Vb.

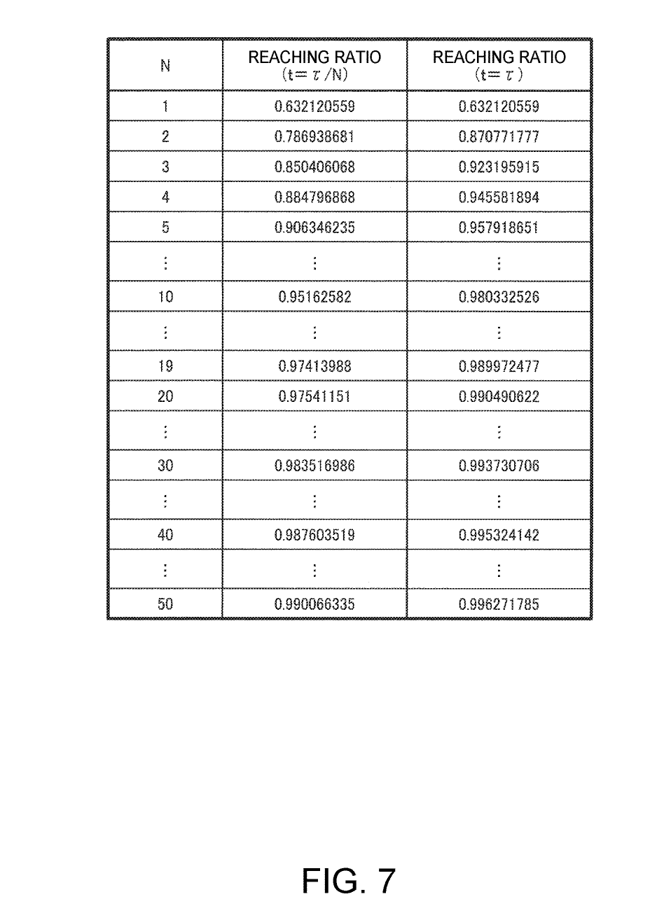

As understood from Expression (5) above and FIG. 7 to be described below, a length T1 of the first period in which a current of N×Ia flows is set to T1=τ/N, a voltage value at t=τ/N does not exceed the desired value Vb and the voltage value for the second period quickly converges on Vb. Here, when an input signal vibrates due to parasitic capacitance or parasitic inductance, there is a possible of an overshoot or an undershoot occurring. This point will be described below in a second embodiment.

Power consumption in the light-emitting unit 110 is a value proportional to a time-integrated value of a current signal. In the scheme of the related art illustrated in FIG. 1, an integrated value is greater than 5×Ia×τ. In the case of the scheme in FIG. 6, “N×Ia×τ/N+Ia×(τ−τ/N)<2×Ia×τ” is satisfied. Thus, the power consumption can be reduced.

As illustrated in FIG. 6, the current signal according to the embodiment is a current signal that has a constant current value for the first period and has a current value greater than a current value for the second period. In the example of FIG. 6, the current value for the first period is constant as N×Ia and is greater than the current value Ia for the second period. In this way, sampling can be performed more stably even for a short light emission period than in the scheme (see FIG. 1) of causing the current value to be constant as Ia for the entire light emission period. Thus, it is possible to reduce the power consumption.

In FIG. 6, the length TL of the light emission period is set to TL=τ and the length T1 of the first period is set to T1=τ/N is set, but the invention is not limited thereto. For example, when τ is the time constant of the filter unit 152, the length TL of the light emission period may be equal to or less than P (where P is positive number equal to or less than 4)×τ. In this way, even when the length TL of the light emission period is the maximum, the length is suppressed to a maximum of 4τ. Therefore, it is possible to further shorten the light emission period than in the scheme of the related art in FIG. 1. As described above, the length TL of the light emission period is also related to power consumption. Therefore, by shortening the light emission period, the power consumption can be expected to be reduced.

However, in the scheme according to the embodiment, the current value for the first period is N×Ia and is greater than Ia which is an original target current. Therefore, when the first period is excessively lengthened, the effect of reducing the power consumption may deteriorate. When the first period is long, the voltage value exceeds the desired value Vb. Thus, there is a concern of an output signal vibrating or a time being taken until convergence on Vb for the second period.

Accordingly, in the embodiment, when TL is the length of the light emission period, the length T1 of the first period may be set to TL/Q (where Q is 2 or more) or less. In this way, the length T1 of the first period is suppressed to a length equal to or less than half of the light emission period TL. Therefore, it is possible to suppress an increase in the power consumption.

The length TL of the light emission period, the length T1 of the first period, and the current value N×Ia (in a narrow sense, the coefficient N) in the first period are not decided singly, but may be decided in consideration of a mutual relation. FIG. 7 is a diagram illustrating a relation of N, a reaching ratio at t=τ/N, and a reaching ratio at t=τ when T1=τ/N. As apparent from FIG. 7, it can be understood that as N is larger, the reaching ratio is closer to 1 at any timing of t=τ/N and t=τ.

For example, when the reaching ratio >0.99 is a condition, a target reaching ratio is not obtained at any timing of t=τ in the case of N<20. That is, when N is relatively small, the length TL of the light emission period may be set to be longer than τ or the length T1 of the first period may be set to be longer than τ/N. Here, the length T1 of the first period is preferably suppressed so that the voltage value for the first period does not exceed Vb.

In contrast, in the case of N≧20, as illustrated in FIG. 7, the reaching ratio at t=τ is greater than 0.99. Accordingly, when T1=τ/N and TL=τ are set, the target reaching ratio can be realized. When N is sufficiently large, the length T1 of the first period or the length TL of the light emission period may be set to be shorter.

In view of the fact that the degree of bluntness of the output waveform is decided according to the time constant τ of the filter unit 152, the length TL of the light emission period or the length T1 of the first period is preferably set using a constant multiple of τ. This is because TL or T1 is set based on τ, and thus the length of the period can be appropriately set even when the characteristics (the time constant τ) of the filter unit 152 are changed. In particular, as expressed in Expression (5) above, the voltage value is a function of the time constant t and converges on N×Vb for the first period. In view of this viewpoint, the length T1 of the first period may be set based on both N and τ. For example, T1 may be τ/N or a constant multiple of τ/N.

As understood from the above description, when the target reaching ratio is different, a set of values of (TL, T1, N) for realizing the target reaching ratio is different. Accordingly, in consideration of the target reaching ratio, the parameters are preferably decided.

An upper limit of N is decided according to hardware characteristics in some cases. For example, in the current supply circuit 132 having the configuration illustrated in FIG. 3, an upper limit of the current value supplied to the light-emitting unit 110 is decided according to an output of the bipolar transistor Tr (the power transistor). In this case, the coefficient N is set in consideration of a target reaching ratio or a relation between of TL and T1 within a range in which the coefficient N does not exceed an upper limit realized by the bipolar transistor Tr. More specifically, the processing unit 160 sets the current setting value so that the current value N×Ia corresponding to the set N is supplied to the light-emitting unit 110.

In this way, in the scheme according to the embodiment, the parameters such as N, TL, and T1 can be set variously. However, at any setting, from the viewpoint of power consumption, when Ia is a current value for the second period, a total current value (a current-integrated value) for the light emission period may be set to be less than a total current value (5×Ia×τ) in a case in which a current signal with the current value which is Ia flows for a period of a length of 5×τ.

The case in which the current signal with the current value which is Ia flows for the period of the length of 5×τ is equivalent to a case in which sampling is performed at a timing equivalent to t=5×τ in the scheme of the related art in FIG. 1. That is, a total current value necessary to perform the stable sampling in the scheme of the related art is 5×Ia×τ. Accordingly, when the total current value can be less than 5×Ia×τ, it is possible to further reduce power consumption than in the scheme of the related art.

In the foregoing embodiment, the current value of the current signal is changed for the first and second periods of the light emission period. Accordingly, as illustrated in FIG. 3, in a case in which the biological information detection device 100 includes the current control unit 130 controlling a current value when the D/A conversion circuit 131 performs the D/A conversion on the current setting value, the D/A conversion period in the D/A conversion circuit 131 is necessarily shorter than the light emission period. Specifically, the D/A conversion circuit 131 performs the D/A conversion on the current setting value for setting a waveform for the light emission period of the light-emitting unit 110 for a D/A conversion period equal to or less than ½ of the light emission period. The D/A conversion period is a period corresponding to an output rate of the D/A conversion circuit 131 and is equivalent to an output interval at which the current setting value which is digital data is subjected to D/A conversion and an analog signal is output.

In this way, it is possible to change the current value for the light emission period at two or more stages, and thus it is possible to realize a current signal waveform indicated by B1 of FIG. 6.

### 3.2 Second Embodiment

In the first embodiment, a current value is N×Ia for the first period and a current value is Ia for the second period. The waveform of the current signal (B1) is steeply changed in the middle of a pulse (the time of transition from the first period to the second period), the output signal waveform is not an ideal form as indicated by B2 of FIG. 6 and becomes a vibrating waveform including an overshoot or an undershoot. Thus, there is a possibility of an extra time being taken for stabilization.

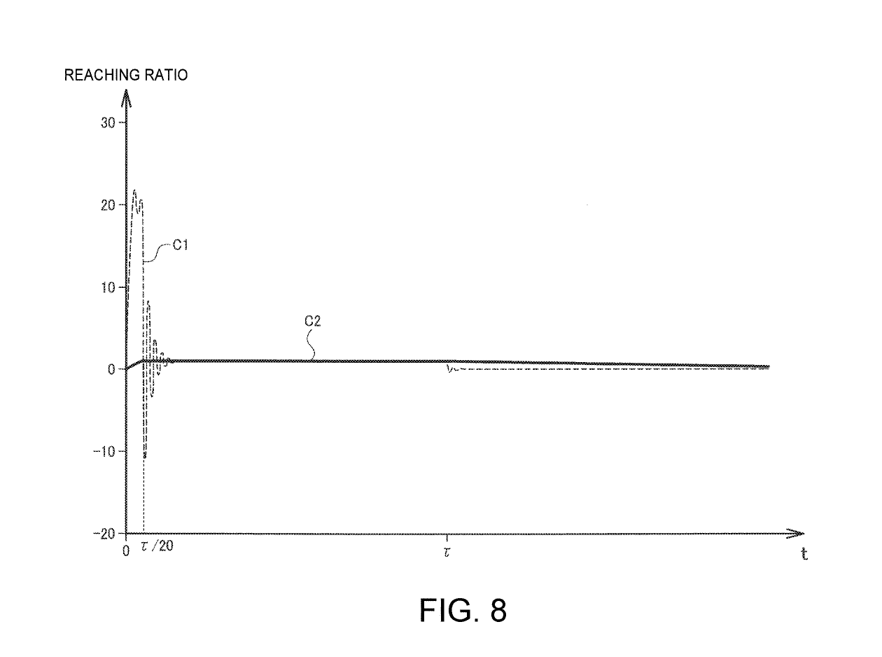

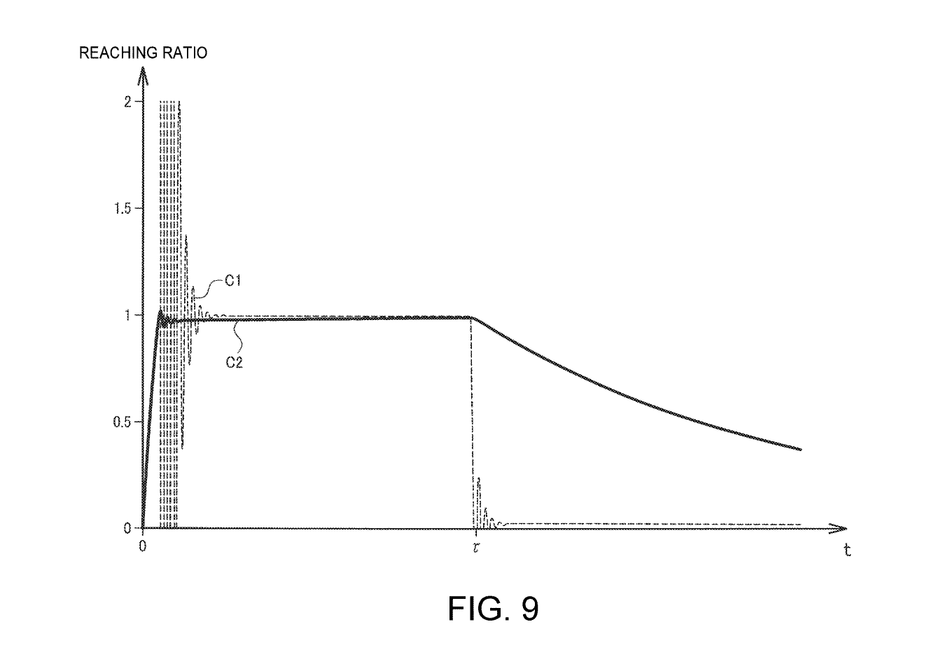

FIG. 8 is a diagram illustrating waveform examples of a current signal and an output signal based on the current signal when there is an influence of parasitic capacitance or parasitic inductance according to the first embodiment. FIG. 9 is a diagram illustrating waveforms expanded in the direction of the vertical axis of FIG. 8. In FIGS. 8 and 9, C1 indicates a current signal waveform and C2 indicates an output signal waveform. In FIGS. 8 and 9, an example of N=20 is illustrated. In FIGS. 8 and 9, the vertical axis represents a reaching ratio, represents a ratio of a current value when Ia serves as a reference in regard to C1, and represents a ratio of a voltage value when Vb serves as a reference in regard to C2.

In FIGS. 8 and 9, the current signal is not stabilized to a target value (in terms of the reaching ratio, N=20 for the first period and 1 for the second period), but is vibrating near the target value. This vibration is caused due to, for example, parasitic resistance, parasitic inductance, or parasitic capacitance of the circuit. As understood from FIGS. 8 and 9, the vibration of the current signal is considerable at a transition timing from the first period to the second period.

As illustrated in FIG. 9, the output signal may also have a vibrating waveform including an overshoot and an undershoot due to the vibration of the current signal. As described above, since it is important how fast to stabilize the output signal in a state in which the output signal is close to a target value (1 at the voltage value Vb and the reaching ratio), the vibration of the output signal can be said not to be preferable.

Accordingly, in the second embodiment, the current is controlled such that the current has a waveform in which high-frequency is enhanced near a rising edge of a light emission pulse. In other words, the current is controlled such that the current signal has a peak value at a predetermined timing in the first period and is a current signal of which a peak value is greater than a current value (in a narrow sense, Ia) for the second period. In addition, a change in the current signal from the peak value to the current value in the second period is to be smoothened.

Specifically, the current signal according to the second embodiment is a current signal in which a current variation value per unit time in falling from the peak value is less than a current variation value per unit time in rising to the peak value. In this way, it is possible to suppress a steep change in the waveform in the middle of a pulse (the time of transition from the first period to the second period). As a result, as in the first embodiment, even when the light emission period is shortened, an output signal after filter processing quickly increases to be stabilized near a desired value. Further, in the scheme according to the embodiment, even when there is an influence of parasitic capacitance or parasitic inductance, a current signal waveform and an output signal waveform after filter processing can be prevented from vibrating.

FIG. 10 is a diagram illustrating waveforms examples of a current signal and an output signal based on the current signal according to the embodiment. D1 indicates a current signal waveform and D2 indicates an output signal waveform. As illustrated in FIG. 10, the current signal has a waveform in which a high frequency is enhanced near rising of a rectangular pulse and is gentler in falling to Ia than the rising. The output signal rises more steeply than in the scheme of the related art of FIG. 1. Therefore, when sampling is performed immediately before t=τ, a substantially stable signal can be sampled near a desired value. The current value at the peak, the length T1 of the first period, and the length TL of the light emission period can be modified variously as in the first embodiment.

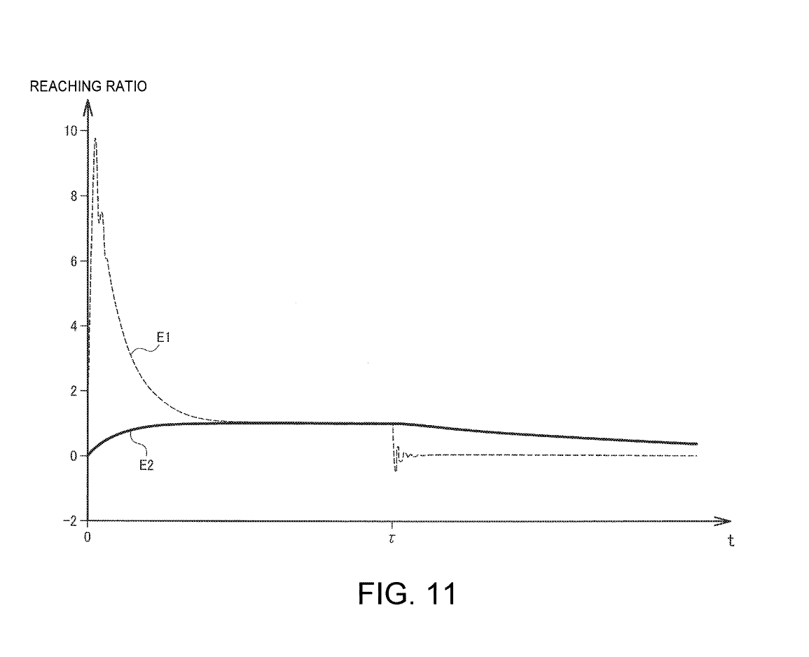

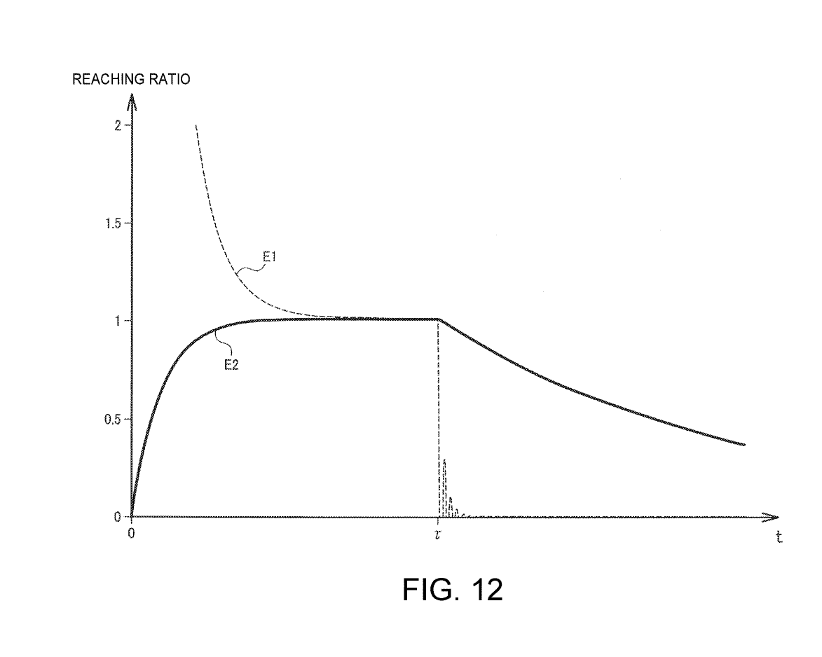

FIG. 11 is a diagram illustrating waveform examples of a current signal and an output signal based on the current signal when there is an influence of parasitic resistance, parasitic capacitance, or parasitic inductance according to the embodiment. FIG. 12 is a diagram illustrating waveforms expanded in the direction of the vertical axis of FIG. 11. In FIGS. 11 and 12, E1 indicates a current signal waveform and E2 indicates an output signal waveform. In FIGS. 11 and 12, the horizontal axis represents a time and the vertical axis represents a reaching ratio, as in FIGS. 8 and 9.

As understood from FIGS. 11 and 12, since a current value from a peak gently decreases in the embodiment, the current signal does not considerably vibrates within the light emission period even in consideration of the parasitic capacitance or the parasitic inductance and the output signal does not vibrate either. Accordingly, more stable sampling can be performed than in the first embodiment.

In the embodiment, a current value is smoothly changed. Therefore, the change in the current value in two stages of N×Ia and Ia as in the first embodiment does not suffice. It is necessary to change the current value more multiple stages (ideally, continuously). For example, when the current value is changed in M stages (where M is an integer equal to or greater than 3), the D/A conversion circuit 131 necessarily outputs D/A conversion results M times within the light emission period. That is, the D/A conversion circuit 131 performs D/A conversion on the current setting value for setting the waveform within the light emission period of the light-emitting unit 110 in the D/A conversion period equal to or less than 1/M of the light emission period. When the D/A conversion period is sufficiently shorter than the light emission period, current signals with various waveforms can be supplied to the light-emitting unit 110 without being limited to the example of FIG. 10.

### 4. Electronic Apparatus

The scheme according to the embodiment can be applied to an electronic apparatus 200 including the biological information detection device 100. Here, the electronic apparatus 200 may be, for example, a wearable apparatus worn by a user.

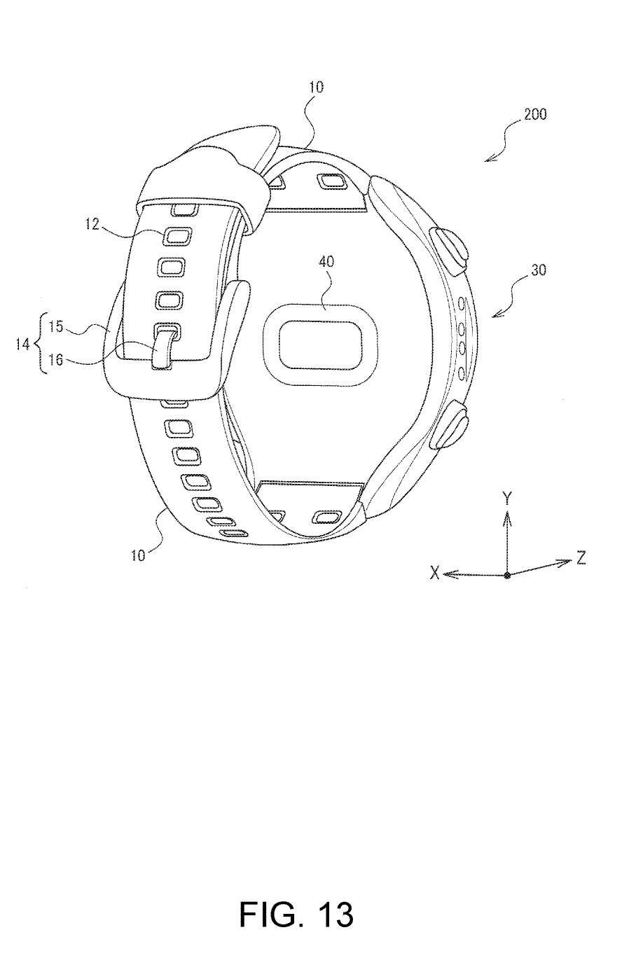

FIG. 13 is a diagram illustrating an example of an outer appearance of a wearable apparatus (the electronic apparatus 200). As illustrated in FIG. 13, the wearable apparatus includes a case unit 30 and a band unit 10 that fixes the case unit 30 to the body (in a narrow sense, a wrist) of the user. Fitting holes 12 and a buckle 14 are provided in the band unit 10. The buckle 14 is configured to include a buckle frame 15 and an engagement portion (protrusion rod) 16.

FIG. 13 is a perspective view illustrating the wearable apparatus in which the band unit 10 is fixed using the fitting hole 12 and the engagement portion 16 when viewed in the direction of the side of the band unit 10 (a surface side which is a subject side in the wearing state among the surfaces of the case unit 30). In the wearable apparatus in FIG. 13, the plurality of fitting holes 12 are provided in the band unit 10. The wearable apparatus is worn on the user by inserting the engagement portion 16 of the buckle 14 into one of the plurality of fitting holes 12. The plurality of fitting holes 12 are provided in the longitudinal direction of the band unit 10, as illustrated in FIG. 13.

A sensor unit 40 is provided in the case unit 30 of the wearable apparatus. In FIG. 13, the sensor unit 40 is assumed to include the light-emitting unit 110 and the light-receiving unit 120. Accordingly, an example in which the sensor unit 40 is provided on the surface which is a subject side at the time of wearing the wearable apparatus in the case unit 30 is illustrated. Here, the provided position of a sensor included in the sensor unit 40 is not limited to the position illustrated in FIG. 13. For example, when the sensor unit 40 includes a body sensor, the body sensor may be provided inside the case unit 30 (in particular, on a sensor substrate included in the case unit 30).

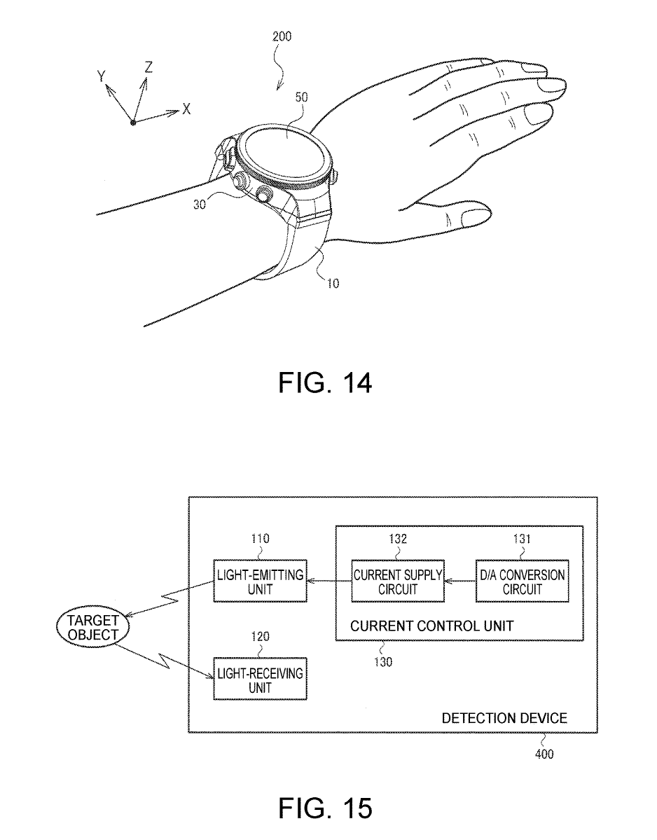

FIG. 14 is a diagram illustrating the wearable apparatus worn on the user when viewed on the side on which a display unit 50 is provided. As understood from FIG. 14, the wearable apparatus according to the embodiment includes the display unit 50 at a position equivalent to a letter plate of a normal wristwatch or a position at which numbers or icons can be viewed. In a state in which the wearable apparatus is worn, the surface of the side of the case unit 30 illustrated in FIG. 13 is in close contact with the subject and the display unit 50 is located at a position at which the user can easily view the display unit 50.

In FIGS. 13 and 14, a coordinate system is set using the case unit 30 of the wearable apparatus as a reference and the positive direction of the Z axis is set to a direction which is a direction intersecting a display surface of the display unit 50 and is directed from rear surface to the front surface when the display surface side of the display unit 50 is set as the front surface. Alternatively, a direction directed from the sensor unit 40 (in a narrow sense, a photoelectric sensor including the light-emitting unit 110 and the light-receiving unit 120 illustrated in FIG. 13) to the display unit 50 or a direction away from the case unit 30 in the normal direction of the display surface of the display unit 50 may be defined as the positive direction of the Z axis. In a state in which the wearable apparatus is worn on the subject, the positive direction of the Z axis is equivalent to a direction directed from the subject to the case unit 30. Two axes perpendicular to the Z axis are X and Y axes. In particular, a direction in which the band unit 10 is mounted on the case unit 30 is set as the Y axis.

Here, the electronic apparatus 200 including the biological information detection device 100 is not limited to the configuration in FIGS. 13 and 14. For example, the electronic apparatus 200 may be a wearable apparatus worn on a part other than an arm. Alternatively, the electronic apparatus 200 may be a portable terminal apparatus such as a smartphone.

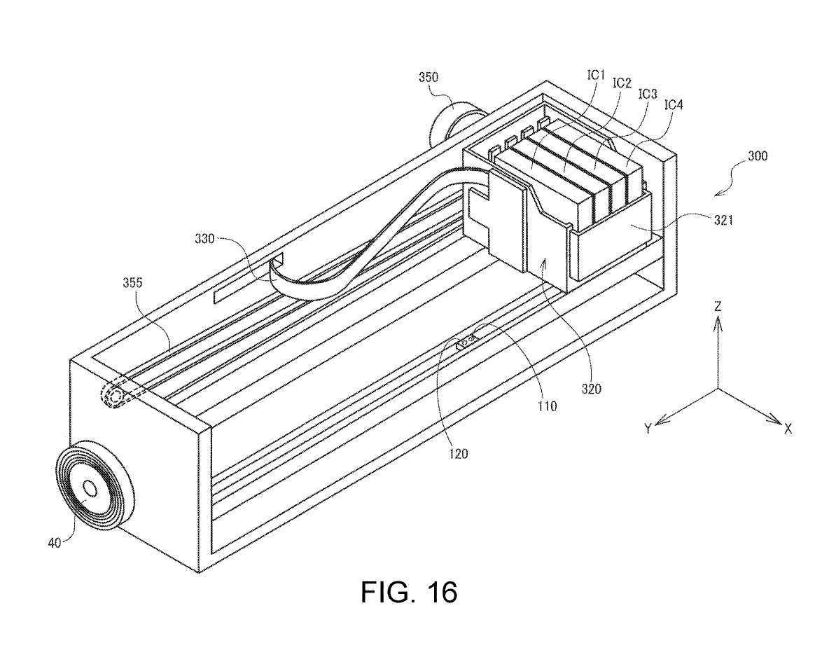

The biological information detection device 100 that detects biological information using the light-emitting unit 110 and the light-receiving unit 120 has been described above, but information detected according to the scheme according to the embodiment is not limited to the biological information. For example, as illustrated in FIG. 15, the scheme according to the embodiment can be applied to a detection device 400 including the light-emitting unit 110 that emits light to a target object, the light-receiving unit 120 that receives reflected light from the target object, and the current control unit 130 that supplies the light-emitting unit 110 with a current signal for causing the light-emitting unit 110 to emit the light. The current control unit 130 of the detection device 400 includes the D/A conversion circuit 131 that performs D/A conversion on a current setting value for setting a waveform for the light emission period of the light-emitting unit 110 for a D/A conversion period equal to or less than ½ of the light emission period and the current supply circuit 132 that outputs a current corresponding to an output voltage of the D/A conversion circuit 131 as a current signal.

The light-emitting unit 110, the light-receiving unit 120, and the current control unit 130 (the D/A conversion circuit 131 and the current supply circuit 132) are the same as the units of the above-described biological information detection device 100. In this way, it is possible to output current signals with various waveforms using the D/A conversion circuit 131 and the current supply circuit 132, and thus it is possible to appropriately detect various physical amounts.

For example, in an example of a printing apparatus (liquid consumption apparatus), whether there is a liquid (a remaining amount of liquid) is detected using a difference between refractive indexes of air and a liquid (ink) which is a consumption target. Alternatively, there is also known a scheme of detecting distance information to a target object using a time-of-flight method or the like of measuring a time in which light radiated from the light-emitting unit is reflected from the target object and is received in the light-receiving unit.

The scheme according to the embodiment can be applied to an electronic apparatus 300 including the detection device 400. The electronic apparatus 300 can be realized by any of various apparatuses. For example, a printing apparatus or a ranging apparatus is considered.

FIG. 16 is a perspective view illustrating main units of a printing apparatus (liquid consumption apparatus) including the detection device 400. The X, Y, and Z axes in FIG. 16 are perpendicular to each other and a front surface direction of the printing apparatus is assumed to be the X direction and a perpendicular direction is assumed to be Z direction at a normal use orientation of the printing apparatus.

The printing apparatus includes ink cartridges IC1 to IC4 (liquid containers or liquid accommodators), a holder 321 that accommodates the ink cartridges IC1 to IC4 to be detachably mounted, a carriage 320, a cable 330, a sheet transport motor 340, a carriage motor 350, and a carriage driving belt 355. In FIG. 16, the light-emitting unit 110 and the light-receiving unit 120 in the detection device 400 are illustrated.

The ink cartridges IC1 to IC4 each accommodate one-color ink (a liquid or a printing material). The holder 321 is mounted so that the ink cartridges IC1 to IC4 are detachably mounted. A head is provided on the surface of the carriage 320 in the −Z direction. The ink supplied from the ink cartridges IC1 to IC4 is ejected from the head to a recording medium. The recording medium is, for example, a printing sheet. The carriage motor 350 drives the carriage driving belt 355 and moves the carriage 320 in the ±Y direction.

The detection device 400 detects ink remaining states of the ink cartridges IC1 to IC4. Specifically, the light-emitting unit 110 radiates light to a prism provided in each of the ink cartridges IC1 to IC4. Then, the light-receiving unit 120 receives reflected light from the prism and converts the reflected light into an electric signal.

For example, when θ1 is a critical angle of total reflection and θ2 is an incidence angle on the prism and the ink remains in the ink cartridge, θ1>θ2 is set to be satisfied. When no ink remains, θ2>θ1 is set to be satisfied. The critical angle θ1 is decided according to the material quality of the prism or the characteristics of the ink.

In this way, when the ink remains, total reflection from the prism does not occur. Therefore, most of the light enters the ink cartridge and a signal received by the light-receiving unit 120 decreases. In contrast, when no ink remains, total reflection from the prism occurs. Therefore, a signal received by the light-receiving unit 120 relatively increases. The detection device 400 detects a remaining amount of ink by detecting a difference in a signal level.

The embodiments and the modification examples to which the invention is applied have been described above. The invention is not limited to the embodiments or the modification examples. In embodiment stages, constituent elements can be modified and embodied within the scope of the invention not departing from the gist of the invention. The invention can be realized in various forms by appropriately combining the plurality of constituent elements disclosed in the embodiments and the modification examples. For example, several constituent elements may be deleted from all of the constituent elements disclosed in the embodiments and the modification examples. Further, the constituent elements described in different embodiments or modification examples may be appropriately combined. In the present specification or the drawings, terms described at least once along with other terms in a broader or identical sense can be replaced with the other terms. In this way, various modifications and applications can be made within the scope of the invention without departing from the gist of the invention.

What is claimed is:

1. A biological information detection device comprising:
a light-emitting unit that emits light to a subject;
a light-receiving unit that receives reflected light or transmitted light from the subject; and
a current control unit that supplies the light-emitting unit with a current signal for causing the light-emitting unit to emit the light,
wherein for a first period of a light emission period of the light-emitting unit, the current control unit supplies the light-emitting unit with the current signal with a current value greater than for a second period which is a period after the first period of the light-emission period.

2. The biological information detection device according to claim 1, further comprising:
a detection unit that performs a process of detecting a signal from the light-receiving unit,
wherein the detection unit includes a filter unit, and
wherein the current control unit supplies the current signal including a frequency component higher than a cutoff frequency of the filter unit to the light-emitting unit for the first period.

3. The biological information detection device according to claim 2,
wherein the filter unit is a lowpass filter or a bandpass filter, and
wherein the cutoff frequency is a cutoff frequency of the lowpass filter or a high-frequency-side cutoff frequency of the bandpass filter.

4. The biological information detection device according to claim 2,
wherein when τ is a time constant of the filter unit, a length of the light emission period is equal to or less than P (where P is a positive number equal to or less than 4)×τ.

5. The biological information detection device according to claim 4,
wherein when Ia is the current value for the second period, a total current value for the light emission period is less than a total current value when the current signal of which the current value is Ia flows for a period with a length of 5×τ.

6. The biological information detection device according to claim 1,
wherein when TL is a length of the light emission period, a length of the first period is equal to or less than TL/Q (where Q is 2 or more).

7. The biological information detection device according to claim 1,
wherein the current signal is a current signal of which the current value is constant for the first period and is greater than the current value for the second period.

8. The biological information detection device according to claim 1,
wherein the current signal has a peak value at a predetermined timing in the first period and is a current signal of which the peak value is greater than the current value for the second period.

9. The biological information detection device according to claim 8,
wherein the current signal is a current signal of which a current variation value per unit time in falling from the peak value is less than a current variation value per unit time in rising to the peak value.

10. The biological information detection device according to claim 1,
wherein the current control unit includes
a D/A conversion circuit that performs D/A conversion on a current setting value for setting a waveform of the current signal for the first and second periods, and
a current supply circuit that outputs a current corresponding to an output voltage of the D/A conversion circuit as the current signal.

11. A detection device comprising:
a light-emitting unit that emits light to a target object;
a light-receiving unit that receives reflected light or transmitted light from the target object; and
a current control unit that supplies the light-emitting unit with a current signal for causing the light-emitting unit to emit the light,
wherein the current control unit includes
a D/A conversion circuit that performs D/A conversion on a current setting value for setting a waveform for a light emission period of the light-emitting unit for a D/A conversion period equal to or less than ½ of the light emission period, and
a current supply circuit that outputs a current corresponding to an output voltage of the D/A conversion circuit as the current signal.

12. An electronic apparatus comprising:
the biological information detection device according to claim 1.

13. An electronic apparatus comprising:
the biological information detection device according to claim 2.

14. An electronic apparatus comprising:
the biological information detection device according to claim 3.

15. An electronic apparatus comprising:
the biological information detection device according to claim 4.

16. An electronic apparatus comprising:
the biological information detection device according to claim 5.

17. An electronic apparatus comprising:
the biological information detection device according to claim 6.

18. An electronic apparatus comprising:
the biological information detection device according to claim 7.

19. An electronic apparatus comprising:
the biological information detection device according to claim 8.

20. An electronic apparatus comprising:
the detection device according to claim 11.
